(19) United States
(12) Patent Application Publication
(10) Pub. No.: US 2017/0189571 A1
(43) Pub. Date: Jul. 6, 2017

(54) DIFFUSER

(71) Applicant: Believe It LLC, Savannah, NY (US)

(72) Inventor: Ryan T. Breen, Savannah, NY (US)

(21) Appl. No.: 15/400,631

(22) Filed: Jan. 6, 2017

Related U.S. Application Data
(60) Provisional application No. 62/275,651, filed on Jan. 6, 2016.

(51) Int. Cl. A61L 9/03; F21V 3/02; F21V 33/00; H04R 1/02

(52) U.S. Cl. CPC A61L 9/03; H04R 1/028; F21V 3/02; F21V 33/008; F21Y 2115/10

(57) ABSTRACT

A diffuser system includes a body with a first opening and a second opening. The first opening forms a recessed cavity and the second opening is configured to diffuse liquid. A vertical member is disposed within the recessed cavity and has at least one ribbed surface and the diffuser system can also include a speaker element and an illuminating mechanism that illuminates the vertical member.

### CROSS-REFERENCE TO RELATED APPLICATIONS

This application claims priority to and the benefit of U.S. Provisional Application No. 62/275,651, filed Jan. 6, 2016, entitled DIFFUSER, the entire content of which is incorporated herein by reference.

### BACKGROUND

1. Technical Field

This disclosure generally relates to diffusers. More specifically, the present disclosure relates to diffusers that can enhance psychological and physical well-being and can include audio and visual capabilities.

2. Related Technology

Diffusers may be used for aromatherapy. Aromatherapy is the practice of using the natural oils extracted from flowers, bark, stems, leaves, roots or other parts of a plant to enhance psychological and physical well-being. Aromatherapy, as indicated by its name, enhances psychological and physical well-being through smell.

In addition to smell, psychological and physical well-being can also be effected by the other four senses: vision, hearing, taste, and touch. For example, hearing a soothing sound, tasting a familiar taste, or touching an object can enhance an individual's psychological and physical well-being. However, purchasing the equipment necessary to affect more than one of the senses can be expensive and time consuming. For example, the equipment may be offered for sale in various stores requiring coordination and time investment to secure all of the equipment. In addition, using the equipment necessary to affect more than one of the senses can occupy a large amount of space that the user may or may not have at her disposal. For example, if an individual lives in a small apartment, amassing the equipment necessary to affect more than one of the senses may not be possible, or it may occupy the majority of the individual's living space.

In some instances, a combination of the senses, rather than just one sense, must be affected to enhance psychological and physical well-being. For example, an individual may need to both hear a soothing sound and smell a pleasant fragrance to have his or her psychological and physical well-being enhanced. Enhancing an individual's well-being can be very beneficial because a high level of well-being can positively impact many aspects of a person's life.

Incorporating functionality to effect more than one of the five senses into a diffuser may be unsightly and reduce the overall aesthetic quality of the diffuser. For example, placing a speaker in a diffuser that is equipped with a visual component may decrease the aesthetic quality of the visual component. Additionally, incorporation of one functionality may reduce the effectiveness of, or preclude the inclusion of, any other functionality. For example, incorporation of a speaker may preclude incorporation of diffusion capabilities due to the limited amount of internal space in the diffuser. Similarly, the liquid-based nature of diffusers may preclude inclusion of electronic components because of safety issues.

Accordingly, there are a number of disadvantages with diffusers that can be addressed.

### BRIEF SUMMARY

Implementations of the present disclosure solve one or more of the foregoing or other problems in the art with systems, methods, and apparatus for using a diffuser system to effect multiple senses (e.g., vision, taste, hearing, smell). In particular, one or more implementations can include a diffuser system having a body with a first opening and a second opening, wherein the second opening is configured to diffuse liquid and the first opening is configured to display a vertical member that can increase the aesthetic value of the diffuser system. The diffuser system can also include a speaker element and a vent.

Diffuser systems can also include a recessed cavity formed by the first opening and the vertical member may be disposed within the recessed cavity.

Diffuser systems can also include an illuminating mechanism that lights a lighting element when desired.

Accordingly, a diffuser system that can effect multiple senses is disclosed.

This Summary is provided to introduce a selection of concepts in a simplified form that are further described below in the Detailed Description. This Summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims, or may be learned by the practice of the disclosure as set forth hereinafter.

### BRIEF DESCRIPTION OF THE DRAWINGS

In order to describe the manner in which the above-recited and other advantages and features of the disclosure can be obtained, a more particular description of the disclosure briefly described above will be rendered by reference to specific embodiments thereof which are illustrated in the appended drawings. It should be noted that the figures are not drawn to scale, and that elements of similar structure or function are generally represented by like reference numerals for illustrative purposes throughout the figures. Understanding that these drawings depict only typical embodiments of the disclosure and are not therefore to be considered to be limiting of its scope, the disclosure will be described and explained with additional specificity and detail through the use of the accompanying drawings in which:

FIG. 1 illustrates a front perspective view of a diffuser system according to an implementation of the present disclosure;

FIG. 2 illustrates a top perspective view of a diffuser system of according to an implementation of the present disclosure;

FIG. 3 illustrates a bottom perspective view of a diffuser system according to an implementation of the present disclosure;

FIG. 4 illustrates a perspective view of the inside of a diffuser system according to an implementation of the present disclosure; and

FIG. 5 illustrates a perspective view of the inside of a diffuser system according to an implementation of the present disclosure; and

FIG. 6 illustrates a perspective view with a cutaway of the inside of a lighting element of a diffuser system according to an implementation of the present disclosure.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments, and is not necessarily intended to limit the scope of the claimed invention.

Unless defined otherwise, all technical and scientific terms used herein have the same meaning as commonly understood by one of ordinary skill in the art to which the present disclosure pertains.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

It will be noted that, as used in this specification and the appended claims, the singular forms “a,” “an” and “the” include plural referents unless the context clearly dictates otherwise. Thus, for example, reference to a “bristle” includes one, two, or more bristles. Similarly, reference to a plurality of referents should be interpreted as comprising a single referent and/or a plurality of referents unless the content and/or context clearly dictate otherwise. Thus, reference to “bristles” does not necessarily require a plurality of such bristles. Instead, it will be appreciated that independent of conjugation; one or more bristles are contemplated herein.

As used herein, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure and/or claimed invention.

Various aspects of the present disclosure can be illustrated by describing components that are bound, coupled, attached, connected, and/or joined together. As used herein, the terms “bound,” “coupled”, “attached”, “connected,” and/or “joined” are used to indicate either a direct association between two components or, where appropriate, an indirect association with one another through intervening or intermediate components. In contrast, when a component is referred to as being “directly bound,” “directly coupled”, “directly attached”, “directly connected,” and/or “directly joined” to another component, no intervening elements are present or contemplated. Furthermore, binding, coupling, attaching, connecting, and/or joining can comprise mechanical and/or chemical association.

To facilitate understanding, like reference numerals (i.e., like numbering of components and/or elements) have been used, where possible, to designate like elements common to the figures. Specifically, in the exemplary embodiments illustrated in the figures, like structures, or structures with like functions, will be provided with similar reference designations, where possible. Specific language will be used herein to describe the exemplary embodiments. Nevertheless it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

The headings used herein are for organizational purposes only and are not meant to be used to limit the scope of the description or the claims.

The present disclosure extends to systems, methods, and apparatus for a diffuser system that can effect multiple senses. In particular, one or more implementations include a diffuser system having a first opening and a second opening, wherein the first opening displays a vertical member and the second opening is configured to diffuse liquid. The vertical member displayed by the first opening may add to the aesthetic value of the diffuser system. The diffuser system can also include a speaker element, a vent for venting heat generated during the diffusion of liquid, a lighting element, and an illuminating mechanism. The system can be configured to effect multiple senses at the same time, including at least vision and hearing.

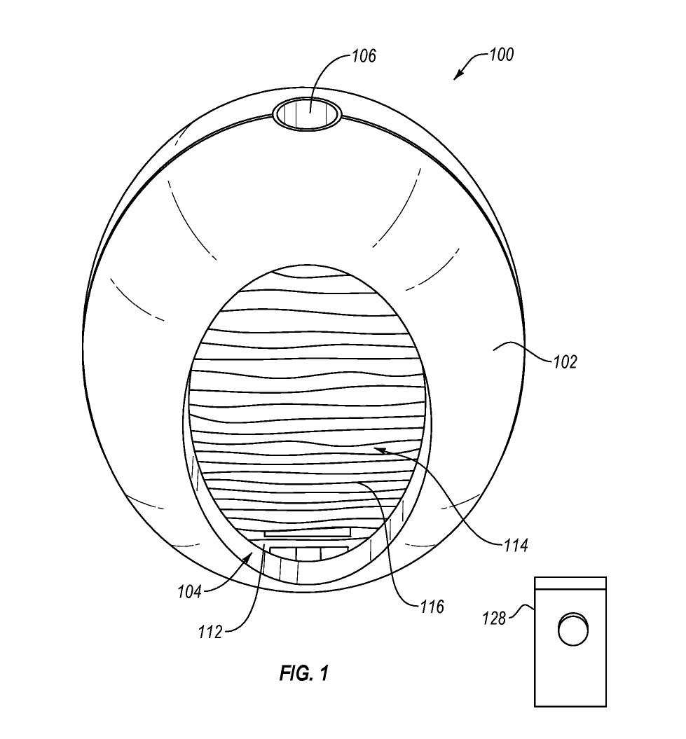

Reference will now be made to the figures of the present disclosure. For example, FIGS. 1-6 illustrate a diffuser system according to an implementation of the present disclosure. The diffuser system 100 comprises a body 102 with a first opening 104, a second opening 106, a speaker element 108, and a vent 110.

The first opening 104 may form a recessed cavity 112 within body 102. As illustrated in FIG. 1, recessed cavity 112 may have a vertical member 114 mounted or otherwise disposed therein. Vertical member 114 may be mounted or otherwise disposed therein in any manner that allows for vertical member 114 to at least partially fit within recessed cavity 112. For example, the perimeter of vertical member 114 may be affixed with glue to the inside of the perimeter of recessed cavity 112. Although the figures show vertical member 114 fully disposed within recessed cavity 112 vertical member 114 may be fully or partially disposed within recessed cavity 112.

In the illustrated embodiment, at least a portion of the vertical member 114 (e.g., a major surface thereof) is visible through the first opening 104. Although the figures show vertical member 114 visible through recessed cavity 112, it should be understood that vertical member 114 need not necessarily be visible through recessed cavity 112. Vertical member 114 may include a ribbed surface 116 or any other type of surface, such as a flat surface, a graduated surface, a smooth surface, or a gritty surface. If vertical member 114 includes a ribbed surface 116, the ribbed surface 116 may add to the aesthetic value of vertical member 114 and therefore to the overall aesthetic value of diffuser system 100. In addition, if vertical member 114 includes a ribbed surface 116, or any other type of friction-creating surface, a liquid, such as water, may flow over vertical member 114 to create a calming sound, such as the sound of water running over rocks in a stream.

Although FIG. 1 depicts vertical member 114 within recessed cavity 112, those skilled in the art will understand that even if the diffuser system 100 does not have a recessed cavity 112 it may still have a vertical member 114. For example, vertical member 114 may be incorporated into the body 102 of diffuser system 100. As an additional example, body 102 may have multiple recessed cavities 112 in which vertical member 114 may be disposed. Accordingly, vertical member 114 is not dependent on the presence of a single recessed cavity 112. In addition, as mentioned previously, vertical member 114 need not have a ribbed surface 116. For example, vertical member 114 may have a flat surface, or any other kind of surface.

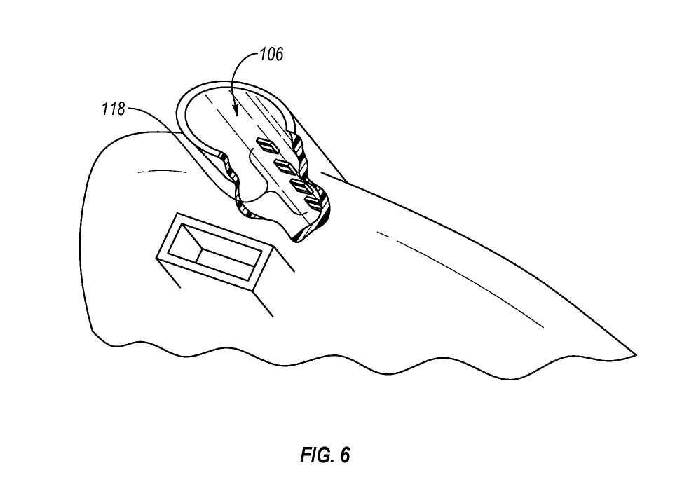

As shown in FIG. 6, the diffuser system 100 may also have a lighting element 118. As shown, lighting element 118 may be embedded within opening 106 and disposed behind vertical member 114. Alternatively, lighting element 118 may be entirely separate from (e.g., disposed behind, below, or to a side of) vertical member 114 and may not be embedded within opening 106. To ensure that the light emitted from lighting element 118 can be seen, vertical member 114 may be transparent or translucent. For example, if lighting element 118 is embedded within vertical member 114, vertical member 114 may be translucent so that light, but not detailed images, may pass through vertical member 114. Alternatively, if lighting element 118 is not embedded within vertical member 114 but is instead disposed separately within body 102 of the diffuser system 100, vertical member 114 may be transparent.

Lighting element 118 may be a single LED or a plurality of LEDs, such as one or more LED arrays. If a plurality of LEDs are used, they may be arranged in, for example, a line, a circle, a square, a flower shape, a rainbow shape, or any other desired shape or arrangement. Lighting element 118 may employ LEDs of the same or different colors. For example, red, white, and blue LEDs could be used during the Fourth of July holiday.

In one preferred embodiment, lighting element 118 may provide sufficient light, i.e., is of a sufficient luminous intensity, to function as a nightlight. That is, lighting element 118 may have a luminous intensity of at least 1300 millicandela (mcd) at 20 milliamps (mA) or at least 5000 mcd at 20 mA. Alternatively, lighting element 118 may have a low nominal luminous intensity on the order of a few hundred mcd. Lighting element 118 may also use multiple LED arrays, each having a specific luminous intensity, either the same or different from each other. If lighting element 118 consists of multiple LED arrays they may be controlled independently or together, to provide an ornamental design.

Those of skill in the art will appreciate that vertical member 114 may be transparent or translucent regardless of whether lighting element 118 is embedded within vertical member 114. Further, lighting element 118 may consist of some other lighting mechanism equivalent to an LED array.

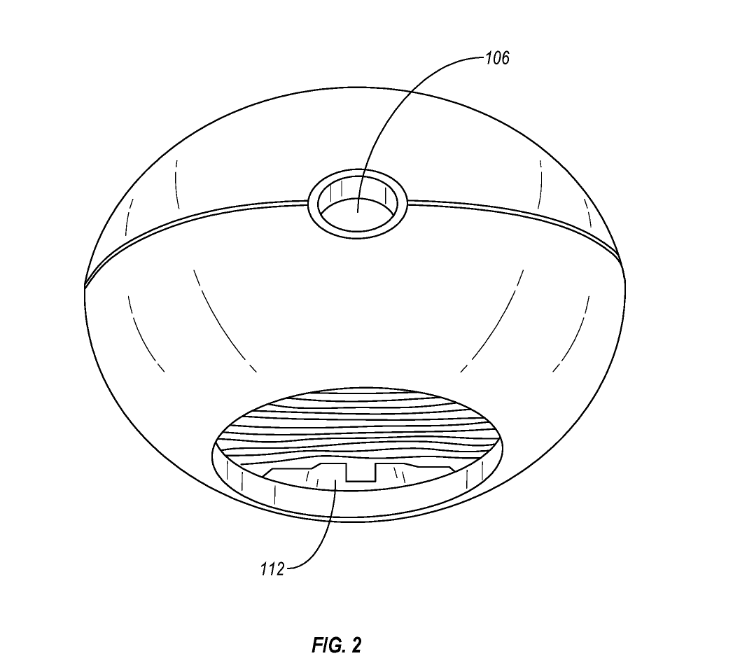

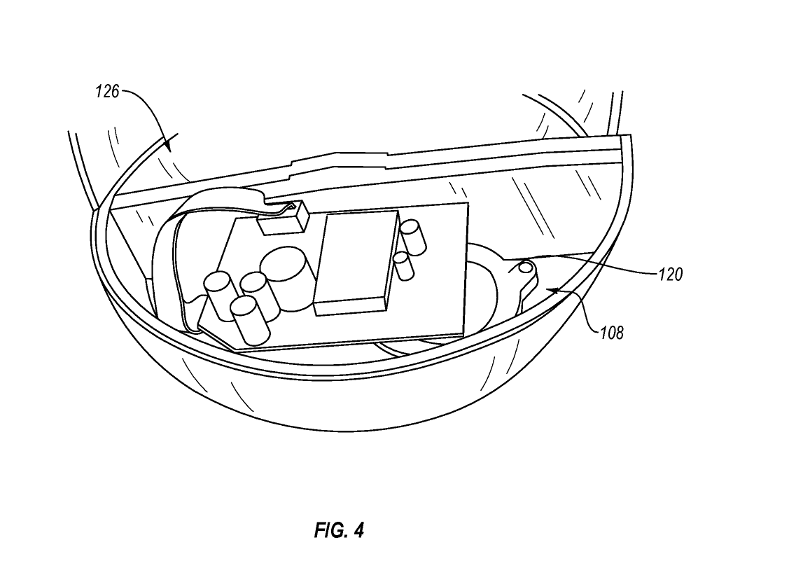

As illustrated in FIG. 2, the diffuser system 100 may have a second opening 106 which may diffuse liquid. For example, diffuser system 100 may include a reservoir 126 (see FIG. 4) to receive and hold the liquid (e.g., water, natural oils, etc.) prior to diffusion. Second opening 106 may diffuse natural oils extracted from flowers, bark, stems, leaves, roots or other parts of a plant. The diffusion of natural oils, or any other liquid, through second opening 106 may enhance the psychological and physical well-being of a user and/or may scent a room. While FIG. 2 depicts second opening 106 in an oval shape, those of ordinary skill in the art will understand that second opening 106 may be of any shape, such as square, circular, triangular, etc. Similarly, those of ordinary skill in the art will understand that second opening 106 may be any size. For example, it may span a small amount of the top of body 102 as shown or it may span the majority of the top of body 102. In addition, while FIG. 2 depicts second opening 106 on the top of body 102 those of ordinary skill in the art will understand that second opening 106 may be disposed on the top, bottom, or any side of body 102. Alternatively second opening 106 may consist of multiple openings.

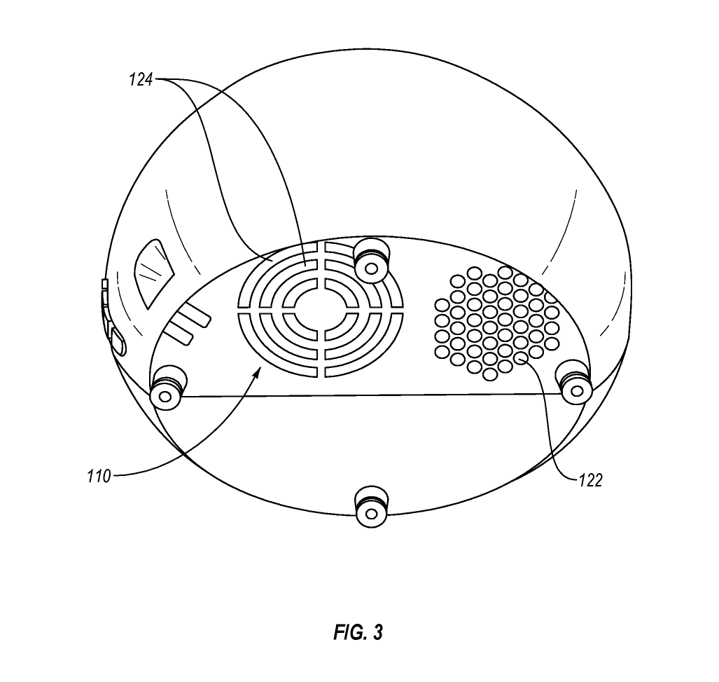

As illustrated in FIGS. 3-5, diffuser system 100 may include a speaker element 108. Speaker element 108 may be comprised of a conventional speaker 120 that is disposed within body 102 and a plurality of holes 122 for releasing the sound made by conventional speaker 120. Those of skill in the art will understand that the plurality of holes 122 may be of any shape and size such that they can reside on body 102. Further, those of skill in the art will understand that speaker element 108 need not be on the bottom of body 102 but instead may be on the top or any of the sides of body 102. Speaker element 108 may be adapted for output of natural sounds, music, animal sounds, etc., stored in the circuit element through the plurality of holes 122 to provide a sound effect.

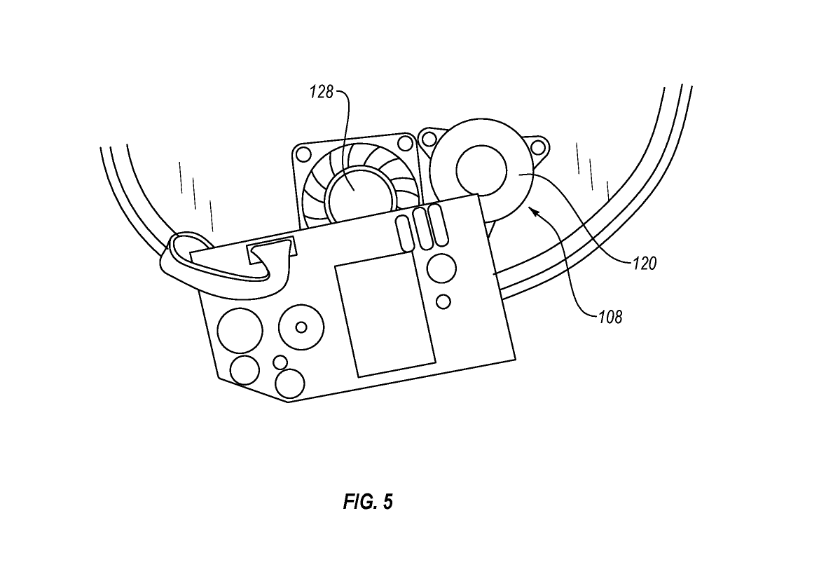

Looking at FIGS. 3 and 5, diffuser system 100 may include a vent 110 that allows hot air generated from the diffusion of liquids by motor 128 to escape body 102. As depicted in FIG. 3, vent 110 may be comprised of a group of cutouts 124 in body 102. Although FIG. 3 depicts vent 110 as being comprised of a group of symmetrical arched cutouts 124, those of skill in the art will understand that vent 110 may be comprised of cutouts of any shape or size or may be comprised of one cutout rather than a group of cutouts. In addition, although FIG. 3 depicts a single group of symmetrical cutouts 124 comprising vent 110, vent 110 may instead consist of multiple groups of symmetrical or non-symmetrical cutouts placed anywhere in body 102.

A wireless infrared remote control may also be used with the present invention. Specifically, diffuser system 100 may have a wireless infrared remote control 128 that sends a light signal to an infrared radiation receiver housed within or on body 102 of diffuser system 100. For example, an infrared radiation receiver may be a small black dot in the center of the right side of body 102. Alternatively, the infrared radiation receiver may be a large dot on the left side of body 102. Those of skill in the art will understand how a standard wireless infrared remote control and infrared radiation receiver communicate. For a more detailed description of an infrared radiation controller see U.S. Pat. No. 4,509,211 to Robbins and U.S. Pat. No. 4,864,647 to Harrington, which are incorporated herein by reference in their entirety. The actual technique used for wireless infrared remote control does not affect the operation of the present invention.

In any event, one will appreciate that implementations of the present disclosure can provide a wide variety of diffuser systems that provide a wide variety of benefits. For example, implementations of the present disclosure can provide a diffuser system having a first opening and a second opening, wherein the second opening is configured to diffuse liquid and the first opening displays a vertical member that adds to the overall aesthetic value of the diffuser system. In addition, certain embodiments can include a speaker element, a vent, a recessed cavity, and a lighting element.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

I claim:

1. A diffuser system, comprising:
a body with a first opening and a second opening, wherein the second opening is configured to diffuse liquid;
a vertical member mounted within the body such that the vertical member is visible through the first opening;
a speaker element; and
a vent for venting heat generated during the diffusion of liquid.

2. The diffuser system of claim 1, wherein the first opening forms a recessed cavity.

3. The diffuser system of claim 2, wherein the vertical member is mounted within the recessed cavity such that water or another liquid can flow over the vertical member.

4. The diffuser system of claim 2, wherein the flow of water is isolated from the speaker element and the lighting element.

5. The diffuser system of claim 1, wherein the speaker element is mounted on the inside of the body.

6. The diffuser system of claim 5, wherein the flow of water is isolated from the speaker element and the lighting element.

7. The diffuser system of claim 1, wherein the vent is comprised of a plurality of holes in the body.

8. The diffuser system of claim 1, further comprising a lighting element that lights the vertical member.

9. The diffuser system of claim 8, wherein the flow of water is isolated from the speaker element and the lighting element.

10. A diffuser system, comprising:
a body with a first opening and a second opening, wherein the first opening forms a recessed cavity and the second opening is configured to diffuse liquid;
a vertical member disposed within the recessed cavity; and
a lighting element that illuminates the vertical member.

11. The diffuser system of claim 10, wherein the vertical member is mounted within the recessed cavity.

12. The diffuser system of claim 10, wherein the vertical member is mounted within the recessed cavity such that water or another liquid can flow over the vertical member.

13. The diffuser system of claim 10, further comprising a speaker element.

14. The diffuser system of claim 13, wherein the speaker element is mounted on the inside of the body.

15. The diffuser system of claim 10, further comprising a vent.

16. The diffuser system of claim 10, further comprising a lighting element that lights the vertical member.

17. The diffuser system of claim 16, wherein the flow of water is isolated from the speaker element and the lighting element.

18. A diffuser system, comprising:
a body with a first opening and a second opening, wherein the first opening forms a recessed cavity and the second opening is configured to diffuse liquid;
a vertical member disposed within the recessed cavity, wherein the vertical member has at least one ribbed surface;
a speaker element, the speaker element comprising
a speaker that is disposed within the body; and
a plurality of holes in the body for releasing the sound made by the speaker;
a wireless infrared remote control for controlling the diffuser system; and
a lighting element that illuminates the vertical member.

19. The diffuser system of claim 18, wherein the vertical member is mounted within the recessed cavity such that water or another liquid can flow over the vertical member.

20. The diffuser system of claim 19, wherein the flow of water is isolated from the speaker element and the lighting element.
